(19) United States
(12) Patent Application Publication
(10) Pub. No.: US 2024/0252420 A1
(43) Pub. Date: Aug. 1, 2024

(54) IMPROVEMENTS IN OR RELATING TO SOAP

(71) Applicants: John TURNER, St Albans, Hertfordshire (GB); Kathryn Robinson, St Albans, Hertfordshire (GB)

(72) Inventors: John TURNER, St Albans, Hertfordshire (GB); Kathryn Robinson, St Albans, Hertfordshire (GB)

(21) Appl. No.: 18/564,362

(22) PCT Filed: May 20, 2022
(86) PCT No.: PCT/EP2022/063728

§ 371 Date: Nov. 27, 2023

(30) Foreign Application Priority Data
May 28, 2021 (GB) 2107627.8

(51) Int. Cl. A61K 8/89; A61K 8/04; A61Q 19/10; C11D 3/37; C11D 3/40; C11D 17/00

(52) U.S. Cl. CPC A61K 8/89; A61K 8/042; C11D 3/373; C11D 3/40; C11D 17/003; A61K 2800/434; A61Q 19/10

(57) ABSTRACT

A soap for hand washing is provided that contains a material that fluoresces when exposed to light such as ultra violet light, the hands are subjected to the light after washing and any fluorescence can indicate inadequate washing, the soap can be used to determine if World Health Organisation guidelines for washing have been adhered to.

### CROSS-REFERENCE TO RELATED APPLICATIONS

The present application claims priority to PCT/EP2022/063728 filed on May 20, 2022 (published as WO 2022/248356 A1 on Dec. 1, 2022), which claims priority to GB 2107627.8 filed on May 28, 2021, the foregoing being incorporated by reference in their entireties for all purposes.

### FIELD

The present invention relates to soaps and in particular to soaps for washing hands. In a preferred embodiment the soap formulation is such that it can be used for monitoring the effectiveness of hand washing and to give an effective visual picture of the extent of the washing. The soap therefore combines the functionality of cleaning and indicating the extent of the cleanliness that is achieved.

### SUMMARY

In a preferred embodiment the soaps of the present invention are suitable for automatic delivery to the hands and have a persistence on the hands whereby the soap can only be removed from the hands by effective washing as required by the particular environment in which the soap is used. The preferred soap formulation of this invention is such that it can remain on the hands for at least 20 seconds and any soap residue remaining on the hands after 20 seconds of washing can provide an indication that the washing has not been effective. The soap therefore enables the user to easily determine if the washing has been sufficient. In particular the soap allows for the assessment of effective washing over the 20 second washing period proscribed by the World Health Organisation guidelines on hand hygiene in health care.

In a further preferred embodiment the soap contains an indicator that can be activated after washing to show that some soap remains on the hands and that further washing is desirable. In particular the soap can contain a light sensitive material which can be activated by shining light onto the washed hands to indicate the presence of residual soap on the hands. Clean hands are safe hands and the soap formulation can be provided with anti-bacterial and/or anti-viral properties as required. Furthermore the soap can be used to enable the user to improve the general effectiveness of their washing activities.

In a preferred embodiment the light sensitive material in the soap is a material that fluoresces when subjected to Ultra Violet (UV) light particularly UVA light. Alternatively the light sensitive material may be chosen to respond when subjected to light from light emitting diodes.

### BACKGROUND

The soap is particularly useful in a system such as those described in our PCT application PCT/EP2021/052748 in which the soap is delivered automatically to the hands by positioning the hands adjacent to a sensor on the soap delivery device. In this embodiment the soap is a liquid or a gel which can be delivered from the device typically by the pumping of a pump that is activated due to the proximity of the hands to the sensor, accordingly in this embodiment the soap should have a viscosity that allows it to be pumped by the device in which it is used. Furthermore, since a preferred use of the soap is in an automatic dispenser it is also important that the soap has a viscosity that enables it to be poured from a container into the dispenser when the soap supply needs to be replenished. A further requirement in this embodiment is that the soap can be manually spread on the hands after delivery and furthermore has a persistence on the hands for at least 20 seconds and that it can only be removed from the hands within a proscribed washing period (such as the 20 seconds proscribed in the WHO guidelines) employing typical hand washing pressures.

Soaps containing light activatable materials are known for example U.S. Pat. No. 6,258,370 discloses a disinfectant that contains an optical brightener which is preferably one that is activated by a UV lamp. The disinfectant can be used for washing hands. According to U.S. Pat. No. 6,258,370 with hand washing higher concentrations of the brightener is used to ensure that some material remains on the hands after rinsing. That patent is not however concerned with ensuring the delivery of the soap, the distribution of the soap on the hands or achieving a desired degree of persistence of the soap on the skin.

United States Patent application publication number 2008/0362893 relates to compositions comprising an ultra violet indicator and uses thereof to visualise and confirm application of a composition to a surface. The compositions are aqueous and may be applied to hard surfaces or skin. The presence of the ultra violet indicator on the surface indicates whether or not the composition has been effectively applied. Similarly United States Patent application publication number 2006/003198 includes additives sensitive to ultra violet light in formulations for topical application to human skin to verify that the formulation has been effectively applied.

PCT applications WO 2006/085907 and WO 2019/032901 relate to a sanitizing composition useful in hand washing which may contain additives sensitive to ultra violet light and the additives can be activated after application to indicate that they are present on the surface with which they are used.

None of these references are concerned with ensuring the soap can be retained on the hands and that the absence of the fluorescence after washing over a proscribed time can be used to indicate that washing has been completed or that the presence of fluorescence indicates inadequate washing. Furthermore, none of the references are concerned with ensuring that the persistence of the soap to the skin over the proscribed time is such that the soap can only be removed by effective washing and rinsing.

### DESCRIPTION

The invention therefore provides a soap comprising a liquid or a gel having a viscosity at ambient temperature that allows it to be delivered from a container to hands when positioned close to an automatic sensor that activates the delivery of the soap from the container and which allows the soap to be easily spread and retained on the hands and furthermore has a persistence on the hands such that the soap can only be removed from the hands by effective washing within a proscribed time; additionally the soap contains a light activatable ingredient which can be activated by exposure to light to indicate the presence of soap residues on the hand after completion of the washing and rinsing.

The proscribed time will be determined by the environment in which the washing is to be performed. Typically the 20 seconds washing as proscribed by the World Health Organisation guidelines is used.

The soap of the present invention is preferably aqueous based and the light activatable material is preferably a material that fluoresces when exposed to UV light particularly UVA light.

The invention further comprises a process for washing comprising placing the skin and nails to be washed adjacent to a proximity sensor that activates the delivery of a soap onto the skin from a container; washing the skin with the soap, rinsing the skin and subjecting the rinsed skin to light wherein the soap contains a light activatable material that is activated by the exposure to the light to indicate if any soap remains on the rinsed hands that are exposed to the light. In a preferred embodiment the process comprises further washing if the exposure to light indicates soap remaining on the rinsed hands.

The light activatable material is preferably a material that fluoresces when exposed to UV light particularly UVA light although light emitting diodes may be used as the source of light and in this embodiment the light activatable material is selected to be responsive to the emitted light.

The term persistence to the skin as used herein requires that at least some of the soap will remain on the skin and/or nails comprising at least part of the hands unless the washing is effective and will also remain on the hands during rinsing of the hands following washing. In this way the subsequent exposure to light such as ultra violet light causes fluorescence where soap remains on the hands indicating incomplete washing. The persistence of the soap on the hands during washing will be governed by the viscosity of the soap and the adhesion of the soap to the skin; it may also depend upon the environment in which the soap is used and the force required for washing. For example, there I can be different requirements for washing in an industrial environment than in a hospital or school environment.

In the preferred embodiment the soap is delivered to the user by pumping from an automatic device and the delivery is activated by placing the hand or hands close to the sensor that activates the pump to deliver soap from the soap container which is preferably part of the device. We have found that in order to be readily pumpable and to be spreadable on human hands the soap should be a liquid or a gel having a viscosity of no more than 8000 centipoise to enable pumping and preferably no more than 1000 centipoise for spreading on hands the viscosity being measured at ambient temperature as measured at 25° C. on a DHR2 TA viscometer fitted with a 40 mm cross-hatched flat plate with an operating gap of 300 μm. In this test samples are exposed to a pre-shear at 1s⁻¹ for 30 seconds followed by a shear rate sweep from 1s⁻¹ to 1000s⁻¹ taking 6 points per decade of shear rate; the shear was applied for 30s at each rate and the viscosity calculated over the fluid 5 seconds of each step. The soap should also have an adhesion as measured by applying a 120 μm thick layer of the soap to a 5 mm thick silicone substrate of shear hardness 0.29 as an average of 5 measurements on an A scale durometer and testing adhesion on a TA.XT+C stable Micro systems texture analyser using a 20 mm diameter probe which was pushed onto the surface of the layer with a 100 gm force for 5 seconds and then pulled off the surface at a speed of 0.1 mm/s and the time to release measured and measuring the strength required to pull it off over 10 cycles. In a preferred embodiment the average force required to move the weight should be not less than 200 grams.

One important aspect of the invention is to check if nails and crevices have been completely and effectively washed or not. Hands have crevices, creases and damage and in instances depending on the age of the individual are of different size and depth and can be difficult to wash well. Nails particularly contain such crevices and use of the soap according to this invention can ensure thorough washing and removal of bacteria germs and viruses to ensure thorough washing.

The World Health Organisation has issued guidelines for effective hand washing with soap and water. The various steps include
i) wet hands
ii) apply soap to cover all hand surfaces
iii) rub palms
iv) rub back of hands
v) rub between fingers
vi) rotational rubbing
vii) rub wrists
viii) rinse hands and dry.

It is recommended that to be effective the process up to step (viii) the rinsing should take at least 20 seconds. The soap of this invention is suitable for use according to these guidelines except it is usually employed on dry hands and the soap of the invention is such that it can be applied to cover all hand surfaces (according to step ii)) and adhere to the hands and nails during step ii). Accordingly, the soap of this invention enables the user to determine if effective washing has been achieved within the 20 second wash proscribed by the World Health Organisation guidelines.

The soap formulation of this invention contains typical ingredients for soap formulations such as thickeners which ensure that the soap has the viscosity required for delivery to the hands and for spreading on the hands as per step ii); it is important that the soap is not too fluid so that it will flow off the hands after application. Additionally the formulation contains a film forming agent that ensures the desired degree of adhesion between the soap and the skin during step ii). If the washing action is sufficient the adhesion of the soap to the hands will be reduced in steps iii) to vii) to allow some soap to be removed and to allow any remaining soap to be removed in step viii). If however the washing action is insufficient the adhesion of the soap to the hands in the area where the washing has been insufficient will remain through step viii) leaving some photosensitiser containing soap on the hands which can be identified by exposing the hands to light such as ultra violet light or the light emitted from a light emitting diode.

The soap formulation of the present invention is preferably an aqueous formulation containing typical conventional soap formulation ingredients. Typically the formulation will contain a detergent which forms the principal cleansing material together with surfactants which can assist with the cleansing and can make the formulation more mild to the skin, a humectant to preserve a moist environment, a preservative to provide storage stability and inhibit microbial growth, and a pH controller together with other incipients such as a fragrance, in a preferred embodiment the formulation is free of alcohols such as ethanol.

In addition the soap formulation of this invention contains a film forming agent that controls the adhesion between the soap and the skin which increases the resistance of the soap to be removed during the hand washing steps of the washing process (the persistence) whereby the soap is removed only by the degree of rubbing required by the particular washing circumstances.

Furthermore the soap formulation of this invention includes a light activatable material and it is important that the light activatable material is contained within the soap and is retained therein during the rubbing steps of the washing process so that it is only removed with the soap when the rubbing is sufficient for the particular washing process. The material also remains within the soap when the rubbing is insufficient and furthermore is not removed from the soap during the rinsing that follows the washing.

Reference to removal of soap from hands and to retention of soap on the hands does not require 100% removal or 100% retention in order for the soap to perform its desired function, it is necessary for sufficient soap to be removed during effective washing that fluorescence is minimised on exposure to light following rinsing. Conversely it is necessary for sufficient soap to remain during washing and rinsing if the washing has been ineffective whereby the fluorescence on exposure to light following rinsing indicates that the washing had been insufficient.

The viscosity of the aqueous soap formulation of this invention should be such that it can be delivered automatically from a dispenser and can be spread and retained on the hands; it should not be so fluid that a substantial amount flows off the hands after application, a thickener can be included to provide the required viscosity; an example of a suitable thickener is sodium chloride. The adherence of the soap to the hands should be such that it can be removed providing the washing action is performed satisfactorily.

As mentioned typical ingredients in conventional handwash formulations include a solvent, one or more detergents, a surfactant, a humectant and a preservative. Depending upon the nature of the ingredients and the particular use envisaged for the soap the pH may need to be adjusted and the thickness or viscosity may need to be adjusted by use of a thickening agent. Furthermore fragrances may be added to provide a desirable odour.

The solvent is usually water.

The detergents are usually alkali metal salts particularly sodium salts of alkyl ether sulphates such as sodium lauryl ether sulphate or sodium isothionate salts such as sodium lauryl methyl isothionate or sodium salts of alkyl sulfonates such as sodium C14-C16 olefin sulfonate. The detergents are typically present in an amount of from 5 to 20 wt % of the formulation. Typical cosurfactants include coamideopropyl betaine, lauryl glucoides and cocamide opropyl betain. The cosurfactant is typically present in an amount from 5 to 10 wt % of the formulations.

Typical humectants when used include glycerine, sorbitant and propylene glycol at levels up to 5 wt % of the formulation. A preservative may be included by way of a biocide to prevent bacteria forming in the soap over time and examples of preservatives that may be used include phenoxyethanol, potassium sorbate, sodium benzoate and ethylhexyl glycerine. When used they are typically employed at levels up to 1 wt % of the formulation. If pH adjustment to lower the pH is required carboxylic acids such as citric acid may be used; whenever if it is desired to raise the pH sodium hydroxide may be used. Where a thickening agent is required typically useful materials include sodium chloride, polyethylene glycol/polypropylene glycol materials such as PEG/PPG-120/10; trimethylpropane trioleate and polyethylene glycol esters such as PEG-18 glyceryl oleate/cocoate.

As indicated the materials listed above are conventional ingredients in handwash formulations. However in order for the soap to have the properties and performance required for the present invention the soap should also include ingredients to provide fluorescence when exposed to light particularly UV light and especially UVA light or light emitted by a light emitting diode. Additionally the soap should include an ingredient which provides the required degree of persistence when it is applied to human skin whereby it can be spread on human hands to have sufficient adhesion that it can only be removed by an effective washing operation.

UV indicators may be any agent that will visibly fluoresce at the concentration used when exposed to radiation having a wavelength of from about 100 nm to about 600 nm, the indicator should be stable and soluble in the compositions of this invention. In some aspects of the disclosure, the UV indicator will fluorescence at longwave UV light (UV-A) of from about 315 nm to about 400 nm. Examples of suitable fluorescent materials that may be used include aluminium hydroxide, phosphorescent zinc sulfide; 4,41-Bis[4-anilino-6-[N-(2-hydroxyethyl)-N-(2-carbonmoyl ethyl) amino-1,3,5-triazine-2-ylamino]-2,2¹ stilbene sulphonic acid disodium salt; isopharone diamine and acridines, cyanines, xanthenes, pyrenes, benzoxazoles, fluoranthenes, phenanthridines, rhodamines, lactones and quinines, salts thereof, and combinations thereof. The UV indicator may suitably be oil soluble or water soluble, such as a water soluble salt.

Examples of suitable skin adhering materials that may be used to provide the required persistence include water soluble polymers such as polyvinylpyrrolidone, acrylate polymers and copolymers; butane diol/vinyl alcohol and vinyl alcohol polymers and copolymers; norbornanediamine cross copolymers.

An example of a suitable formulation is

Formulation A

| | Formulation | |
|---|---|---|
| Ingredients | % | Function |
| Deionized water | To 100 | solvent |
| Sodium C14-16 olefin sulfonate | 22 | detergent |
| Cocamide methyl MEA | 3.5 | cosurfactant/thickening |
| | | agent |
| Coamideopropyl betaine | 8 | cosurfactant |
| Glycerin | 0.8 | humectant |
| Disodium EDTA | 0.1 | preservative |
| Citric acid | 0.115 | pH adjustment |
| Parfum | 0.7 | fragrance |
| Sodium chloride | 0.6 | thickening agent |
| Ethylhexylglycerin, | 0.6 | preservatives |
| phenoxyethanol | | |
| Polyvinyl pyrolidone | 0.5 | skin film forming agent |
| Elara EL-00 Aluminium | 2 | fluorescent |
| hydroxide | | |

Tests were performed on various sample formulations to illustrate how the viscosity of the soap and its adhesion to surface can be adjusted by varying the composition as is shown in the attached Figures.

It was found that formulation A could be readily delivered from a device according to Application PCT/EP2021/052748 by a pump within the device operated by placing hands adjacent to a sensor whereas it was also found that Formulation A could be spread over hands and that it had a greater persistence on the skin during washing providing fluorescence when used according to PCT/EP2021/052748.

Because of the differing circumstances under which the soap of this invention will be used it is not possible to specify exact values and limits for viscosity and adhesion however various formulations were developed to illustrate that the skilled person given the information provided herein would be able to develop a soap having the properties required to perform the invention.

For instance in order to obtain a soap having the viscosity required for delivery, spreading on particular hands and viscosity/shear profiling which entails subjecting a material to a range of shear conditions and observing its viscosity throughout. From the resulting “flow curve” viscosity at any relevant shear rates or stresses and the degree of non-Newtonian (typically shear thinning) behaviour exhibited by a material can be identified and quantified.

Controlled rate viscosity profiles, where shear rate is swept, typically across mid to high shear rates, are good for obtaining a rapid viscosity profile to correlate to a range of handling conditions, particularly where a material is forced to flow at certain rates through the action of pumps, coating equipment or manually applied forces.

Zero-shear viscosity is the plateau viscosity often seen as shear rate is reduced towards zero. It serves, therefore, as a measure of the effective viscosity in an at-rest condition. Because of this, zero-shear viscosity of a multi-phase formulation—and more significantly, of the continuous phase in such a system—is a significant contributor to stability against sedimentation or creaming a high value resisting movement of the dispersed phase.

The following other Formulations were developed and tested.

Formulation B

| Ingredients | Formulation % |
|---|---|
| Deionized Water | To 100 |
| Sodium C 14-16 Olefin Sulfonate | 22 |
| Cocamide Methyl MEA | 3.5 |
| Coamidopropyl Betaine | 8 |
| Glycerin | 0.8 |
| Polyquaternium-7 | 0.5 |
| Disodium EDTA | 0.1 |
| Citric Acid | 0.115 |
| Parfum | 0.7 |
| Sodium Chloride | 0.6 |
| Ethylhexylglycerin, Phenoxyethanol | 0.6 |
| PVP K30 | 0.9 |
| Elara EL-00 | 2.3 |
| Salicylic Acid | 0.5 |
| Propylene Glycol | 1.2 |

Formulation C

| Ingredients | Formulation % |
|---|---|
| Deionized Water | To 100 |
| Sodium C 14-16 Olefin Sulfonate | 8.36 |
| Cocamide Methyl MEA | 3.5 |
| Coamidopropyl Betaine | 2.4 |
| Glycerin | 0.8 |
| Disodium EDTA | 0.1 |
| Citric Acid | 0.115 |
| Parfum | 0.7 |
| Sodium Chloride | 1.26 |
| Ethylhexylglycerin, Phenoxyethanol | 0.6 |
| Norbornanediamine/Resorcinol Diglycidyl | 0.8317 |
| Ether crosspolymer | |
| Elara EL-00 | 0.1389 |
| Aluminium Hydroxide | 0.0294 |
| Olea Europaea Fruit Extract | 0.01 |

The viscosities and the adhesions of the formulations was measured using the test methods described herein and the results are shown in the attached Figures in which

### BRIEF DESCRIPTIONS OF THE DRAWINGS

FIG. 1 shows the viscosities of Formulations A and C.

FIG. 2 is the stress vs shear plots for Formulation A and C.

FIG. 3 shows the Peak force required to remove the probe in the adhesion test for Formulations A and C.

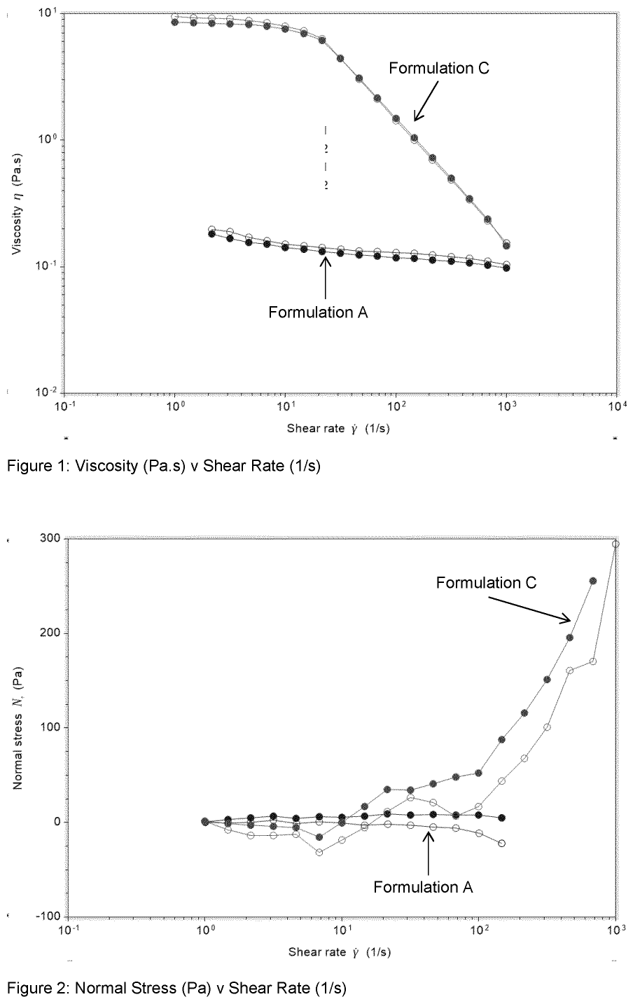

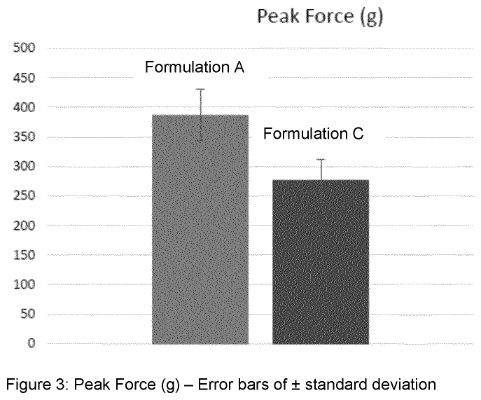

Formulation A was tested for hand washing in the device described in our PCT application PCT/EP2021/052748 and it was found that after vigorous washing for 20 seconds followed by rinsing no fluorescence remained on the hands whereas with less vigorous washing for a shorter time there was considerable fluorescence on the hands.

1. A soap comprising a liquid or a gel having a viscosity at ambient temperature that allows it to be delivered to hands when positioned close to an automatic sensor that activates the delivery of the soap and which allows the soap to be easily spread on the hands and furthermore has a persistence on the hands such that the soap can only be removed from the hands by effective washing; additionally the soap contains a light activatable ingredient which can be activated by exposure to light to indicate the presence of soap residues on the hand after completion of the washing and rinsing; wherein a liquid or a gel has a viscosity of no more then 1000 centipoise at ambient temperature as measured at 25° C. on a DHR2 TA viscometer filled with a 40 mm cross-batched flat plate with an operating gap of 300 μm.

2. The soap according to claim 1, which is aqueous based.

3. The soap according to claim 2, in which the light activatable material is a material that fluoresces when exposed to UV light particularly UVA light.

4. The soap according to claim 1, containing a thickener which ensures that the soap has the viscosity required for delivery to the hands and for spreading on the hands.

5. The soap according to claim 4, containing a film forming agent that provides the desired degree of adhesion between the soap and the skin.

6. A process for washing comprising placing the skin and nails to be washed adjacent to a proximity sensor that activates the delivery of a soap onto the skin; washing the skin with the soap, rinsing the skin and subjecting the rinsed skin to light wherein the soap contains a light activatable material that is activated by the exposure to the light to indicate if any soap remains on the rinsed hands that are exposed to the light, in which the soap is a liquid or a gel having a viscosity of more than 1000 centipoise at ambient temperature as measured at 25° C. on a DHR2 TA viscometer filled with a 40 mm cross-hatched flat plate with an operating gap of 300 μm.

7. The process according to claim 6, in which the light activatable material is a material that fluoresces when exposed to UV light particularly UVA light.

8. The process according to claim 7, in which the soap is delivered to the user by pumping from an automatic device.

9. The process according to claim 8, in which the delivery is activated by placing the hand or hands close to the sensor.

10. (canceled)

11. The process according to claim 10, in which the soap has an adhesion as measured by applying a 120 μm thick layer of the soap to a silicone substrate and testing adhesion on a TA.XT+C stable Micro systems texture analyser using a 20 mm diameter probe which was pushed onto the surface of the layer with a 100 gm force for 5 seconds and then pulled off the surface at a speed of 0.1 mm/s and the time to release measured and measuring the strength required to pull it off over 10 cycles of not less than 200 grams.

12. The process according to claim 11, wherein effective washing is demonstrated if the exposure to light indicates effective washing after 20 seconds.

13. The process according to claim 12, comprising further washing the hands if the exposure to the light indicates the presence of soap residues after completion of the washing and rinsing.
